(19) United States
(12) Patent Application Publication
(10) Pub. No.: US 2024/0086761 A1
(43) Pub. Date: Mar. 14, 2024

(54) NEUROERGONOMIC API SERVICE FOR SOFTWARE APPLICATIONS

(71) Applicant: Microsoft Technology Licensing, LLC, Redmond, WA (US)

(72) Inventors: Aashish PATEL, San Diego, CA (US); Weiwei YANG, Seattle, WA (US); Hayden HELM, San Francisco, CA (US); Daniel J. MCDUFF, Seattle, WA (US); Siddharth SIDDHARTH, Redmond, WA (US); Jen-Tse DONG, Bellevue, WA (US)

(73) Assignee: Microsoft Technology Licensing, LLC, Redmond, WA (US)

(21) Appl. No.: 17/944,022

(22) Filed: Sep. 13, 2022

(51) Int. Cl. G06N 20/00; A61B 5/16; G06F 3/01; G06K 9/62

(52) U.S. Cl. CPC G06N 20/00; A61B 5/165; G06F 3/015; G06K 9/6256

(57) ABSTRACT

The present concepts include a neuroergonomic service that processes multimodal physiological, digital, and/or environmental inputs from a user and predicts cognitive states of the user. Thus, the neuroergonomic service provides personalized feedback to the user about her current mental and physiological wellbeing to enable modulation of mood, stress, attention, and other cognitive measures for improved productivity and satisfaction. The neuroergonomic service utilizes machine learning models that are trained offline using sensor inputs taken from participants in a controlled environment that purposefully induce an array of cognitive states upon the participants.

### BACKGROUND

Neuroergonomics is a field of study that applies the principles of neuroscience (the study of the nervous system using physiology, biology, anatomy, chemistry, etc.) to ergonomics (the application of psychology and physiology to engineering products). For example, neuroergonomics includes studying the human body, including the brain, to assess and improve physical and cognitive conditions. The potential benefits of using neuroergonomics can include increased productivity, better physical and mental health, and improved technological designs.

### SUMMARY

The present concepts include a service that is capable of leveraging multimodal physiological, digital, and environmental inputs to provide broadly applicable neuroergonomic insights to users and applications. The multimodal inputs may be derived from various user interactions, including internal interactions (cognition), individual-environment interactions (actions), and environment-individual interactions (responses). Based on such inputs, the service can provide personalized feedback to the user to enable modulation of mood, stress, attention, and other cognitive measures for improved productivity and satisfaction.

### BRIEF DESCRIPTION OF THE DRAWINGS

The detailed description below references accompanying figures. The use of similar reference numbers in different instances in the description and the figures may indicate similar or identical items. The example figures are not necessarily to scale. The number of any particular element in the figures is for illustration purposes and is not limiting.

FIG. 1 illustrates an example use case scenario of a neuroergonomic service, consistent with some implementations of the present concepts.

FIG. 2 illustrates an example neuroergonomic system, consistent with some implementations of the present concepts.

FIG. 3 illustrates an example data flow, consistent with some implementations of the present concepts.

FIG. 4 illustrates an example timeline of tasks, consistent with some implementations of the present concepts.

FIG. 5 illustrates an example neuroergonomic method, consistent with some implementations of the present concepts.

FIG. 6 illustrates an example configuration of a neuroergonomic system, consistent with some implementations of the present concepts.

### DETAILED DESCRIPTION

### Technical Problem

Employers and employees alike (as well as schools and students, and even individuals) would like to increase productivity without harming people's mental wellbeing. The world is moving towards a more hybrid work environment where students engage in virtual learning from home and employees work from home, hotels, airports, etc. Such a multipurpose environment means that people are facing more distractions, multitasking more than ever, and juggling more demanding schedules without defined work time and personal time. These factors can lead to job dissatisfaction, increased stress, poor health, and other problems.

Accordingly, there is a need for a comprehensive solution that passively assesses the user's mental and physical wellbeing using physiological and neurological data and automatically mitigates adverse psychological impacts of dissatisfaction, stress, fatigue, mental overload, anxiety, burnouts, mental breakdowns, etc. Such a solution would be able to correlate many modalities of input signals in a holistic manner to the person's mental wellbeing and make sense of all of the inter-relationships among the sensed signals and between the signals and the cognitive states. The solution would ingest multimodal signals, generate a model to process the signals, and output useful neuroergonomic insights, while also providing integration points for applications to be developed for using, displaying, and taking actions based on the neuroergonomic insights. Such biosignal-informed neuroergonomic insights can be used to modulate the user's workload, control her schedule, change her environment to provide more satisfying experience, and improve the user's wellbeing.

### Overview

The present concepts provide a technical solution to the above problems. A neuroergonomic service can capture real-time neurological, physiological, and/or biological signals from a user with her permission. The signals include one or multiple sensing modalities. For example, contact sensors measure the user's heart rate or use skin conductance to measure perspiration. As more examples, non-contact sensors measure the user's pupil size or facial expression. Not only can the user permit the collection of these inputs but also can permit the use of the collected input data by the neuroergonomic service to process these signals and make intelligent decisions about the user's state, such as how the user is feeling at the time. These input signals allow the neuroergonomic service to determine and present a holistic view of the user as a person.

The neuroergonomic service outputs various types of neuroergonomic indicators, such as cognitive load, affective state, stress, and attention. Cognitive load indicates the user's mental effort expended (or the amount of mental resources needed to perform a task) and thus indicate how busy the user's mind is. For example, the user's mind may be fatigued from overusing her mental working memory resources, particularly from long-term mental overload. The affective state indicates whether the user's level of arousal is high or low and indicate whether the user's valence is positive or negative. For example, high arousal and negative valence means that the user is anxious, fearful, or angry. High arousal and positive valence means that the user is happy, interested, joyful, playful, active, excited, or alert. Low arousal and negative valence means that the user is bored, sad, depressed, or tired. Low arousal and positive valence means that the user is calm, relaxed, or content. Stress indicates the user's level of emotional strain and pressure that the user is feeling in response to events or situations. Attention indicates the user's level of mentally concentrating on particular information while ignoring other information. This level of focalization of consciousness also indicates how easily the user's mind might be distracted by other stimuli, tasks, or information. Other cognitive states of the user are possible with the present concepts.

The neuroergonomic indicators can be output to the user to provide insights into her neurological state. Alternatively or additionally, the neuroergonomic indicators can be output to an application. The neuroergonomic service can be a backend service that an application can leverage to change its settings or change the environment of the user based on the output from the neuroergonomic service. Based on the output from the neuroergonomic, the digital environment of the user can be adapted to meet the neurological needs of the user, for example, by changing the user interface elements such as screen brightness, font size, audio volume, etc. The application can also display the multimodal inputs and/or the neuroergonomic indicators to the user.

The cognitive states of the user can affect the user's performance, health, attitude, satisfaction, etc. Thus, gaining valuable insight into and managing the user's cognitive states can be important for managing a workplace and managing individual workload and productivity. The present concepts can enable modulation of the user's mood, improve productivity, reduce stress and fatigue, and improve satisfaction.

### Scenario

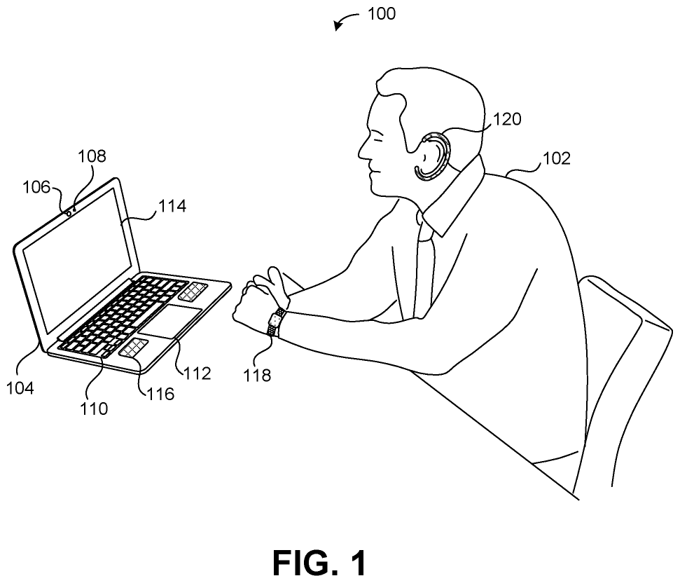

FIG. 1 illustrates an example use case scenario 100 of a neuroergonomic service, consistent with some implementations of the present concepts. In this use case scenario 100, a user 102 is working remotely from home and using a laptop 104 to participate in a virtual meeting by running a videoconferencing application on the laptop 104.

The laptop 104 includes a camera 106 that the videoconferencing application can use to capture images of the user 102. Those captured images are transmitted to other participants in the virtual meeting. The laptop 104 also includes a microphone 108 that the videoconferencing application can use to capture audio, such as the user's speech, which is transmitted to other participants in the virtual meeting. The laptop 104 also includes a keyboard 110 and a touchpad 112 that the user 102 can use to provide input. The laptop 104 also includes a display 114 for showing graphics to the user 102, including the videoconferencing application's interface. The laptop 104 also includes a speaker 116 for outputting audio, such as speech of other participants in the virtual meeting.

In the use case scenario 100, the neuroergonomic service processes multiple modes of input signals, uses machine learning models to determine the cognitive states of the user 102, and enables applications to effect changes to reduce fatigue and increase productivity.

For example, the multiple modes of inputs that affect the user 102 are sensed and measured. The user 102 can choose to opt in to have the camera 106 capture the user's pupil size, facial expressions, heart rate, and/or breathing rate. The camera 106 can sense the ambient light in the user's environment. The user 102 can choose to permit the microphone 108 to capture the background audio in the user's environment. The user 102 can wear a smartwatch 118 or any other wearable devices, and choose to take one or more measurements. The smartwatch 118 can measure the user's heart rate, perspiration rate, blood pressure, body temperature, body fat, blood sugar, etc. The smartwatch 118 can include an inertial measurement unit (IMU) that measures the user's motions and activities, such as being asleep, sitting, walking, and running. The user 102 can choose to wear an electroencephalogram (EEG) sensor 120. Depending on the type, the EEG sensor 120 may be worn around the scalp, behind the ear (as shown in FIG. 1), or inside the ear. The EEG sensor 120 includes one or more electrodes that measure electrical activities of the user's brain. With the user's permission, the laptop 104, in conjunction with the operating system and/or applications, can measure usage telemetry, such as typing rate, clicking rate, scrolling/swiping rate, etc., and also provide the digital focus of the user 102 (e.g., reading, watching, listening, composing, etc.).

With the user's permission and knowledge, these physiological inputs, environmental inputs, and digital inputs enable the neuroergonomic service to use trained machine learning models to predict the cognitive states of the user 102. The neuroergonomic service can output a comprehensive neuroergonomic view of the user 102. The outputs from the neuroergonomic service can include the sensed measurements (e.g., heart rate, pupil size, etc.) and/or the cognitive state predictions (e.g., stress, attention, etc.).

The neuroergonomic service includes general machine learning models that can be used to predict the states of any user. Additionally or alternatively, the neuroergonomic service includes individualized machine learning models that are specifically trained to predict the state of the user 102. Such user-specific machine learning models are not only better at interpreting signals from the user 102 and predicting the cognitive states of the user 102 but also able to learn the daily, weekly, or monthly rhythm of the user 102. For example, the user 102 may drink coffee every morning and has very high focus level in the mornings for high productivity, whereas the user 102 usually eats a large lunch and is drowsy with low attention level in the early afternoons. The individualized machine learning models can be fine-tuned to the user's specific patterns and signals. Applications can leverage the insights of the user's cognitive states provided by the neuroergonomic service to better customize the user's workflow.

Many uses of the neuroergonomic insights provided by the neuroergonomic service are possible. One or more applications, including the videoconferencing application that the user 102 is using, can access and use the outputs from the neuroergonomic service for a myriad of purposes. For example, if the videoconferencing application detects an elevated cognitive load condition among one or more of the participants in the virtual meeting based on the outputs from the neuroergonomic service, then the videoconferencing application can recommend a short break or recommend rescheduling the virtual meeting to continue at a later date and time. If the videoconferencing application determines through the neuroergonomic service that the user 102 is under a lot of stress, then the videoconferencing application may recommend activities to reduce or eliminate stressors to lower the user's stress level.

As further examples, the operating system of the laptop 104 can automatically and seamlessly enter a focus mode during a deep work block where the attention level of the user 102 is low. The operating system can take any number of actions depending on the cognitive states of the user 102. For example, the operating system can adjust the display brightness, change the theme (e.g., the background wall paper, window and menu colors, cursor size, font size, etc.), produce ambient sounds (e.g., meditative audio), etc., to improve the cognitive states of the user 102 (e.g., increase attention level or reduce stress level).

Many other uses of the neuroergonomic service are possible. For example, an application can use the neuroergonomic service for neuromarketing and/or neuroesthetics. Neuromarketing applies neuropsychology to marketing by studying how consumers' cognitive states are affected by marketing stimuli. Neuroesthetics studies how aesthetics affect neuropsychology, for example, how people's perception of art affects their feelings. An application can use the cognitive state insights from the neuroergonomic service to run more effective advertisements, present more desirable products, display more pleasing graphical user interfaces, and implement other strategies that are more in tune with the user's preferences, motivations, and decision-making factors.

### System

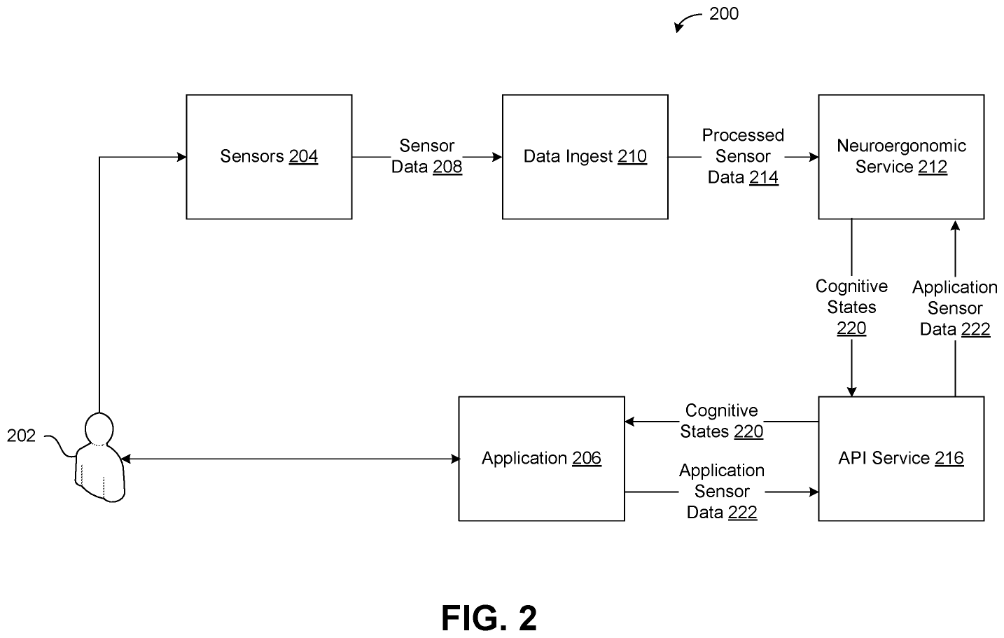

FIG. 2 illustrates an example neuroergonomic system 200, consistent with some implementations of the present concepts. FIG. 2 shows a user 202. The cognitive states of the user 202 are affected by and/or exhibited by biological, neurological, and/or physiological conditions.

These conditions of the user 202 are measured by one or more modalities with the user's consent. As mentioned above, the user's internal cognitive interactions, the user's actions upon her environment, and the user's responses to her environment can be measured.

For example, the neuroergonomic system 200 includes sensors 204 that measure the conditions associated with the user 202. The sensors 204 can include biosensors and/or environmental sensors. Biosensors can be contact sensors, such as an electroencephalogram (EEG) sensor that monitors brain activity, a functional near-infrared spectroscopy (fNIRS) sensor that monitors brain activity, an electrocardiogram (ECG) sensor that measures heart activity, a thermometer that measures body temperature, a perspiration sensor, a respiration sensor that measures breathing rate, etc. Biosensors can be contactless sensors, such as a photoplethysmography (PPG) sensor for measuring the heart rate and the breathing rate; an infrared camera for measuring the body temperature; or a red-green-blue (RGB) camera for eye tracking, measuring pupil dilation, recognizing facial expressions, or detecting skin flushing or blushing. Environmental sensors can include a camera for detecting ambient light or a microphone for detecting ambient sounds. The sensors 204 output sensor data 208.

The sensors 204 can be standalone devices, such as discrete hardware products. Or, the sensors 204 can be integrated into existing devices. For example, a virtual reality (VR) headset can include multiple sensors, such as an EEG sensor, a thermometer, a perspiration sensor, a camera for measuring pupil dilation, a microphone, etc. A keyboard or a touchpad can include a finger pulse heart rate monitor. And an earphone for playing audio can also include an in-ear EEG sensor.

The conditions of the user 202 can also be measured by an application 206. The application 206 can act as a sensor to detect the type of digital activity that the user 202 is engaged in (e.g., watching a movie, reading the news, or drafting a work email), the intensity of the activity (e.g., slowly scrolling through social media or typing 100 words per minute), and/or the number of activities. The application 206 outputs application sensor data 222.

The data collected by sensing the condition of the user 202 may be inputted to a neuroergonomic service 212. In one implementation, the sensor data 208 from the sensors 204 and the application sensor data 222 from the application 206 (e.g., the multimodal signal data sources and/or streams) are aggregated to a cloud endpoint, processed, and then inputted to the neuroergonomic service 212.

The sensor data 208 from the sensors 204 are processed in a data ingest 210. The data ingest 210 includes scripts and/or pipelines that process the sensor data 208 and output processed sensor data 214. Alternatively or additionally, the data ingest 210 includes hardware-implemented processing, for example, on embedded systems in the sensors 204. The sensor data 208 can have different formats or use different time scales. The data ingest 210 can remove bad inputs, perform temporal alignment of multiple signals, and format and/or structure the sensor data 208 into processed sensor data 214 that the neuroergonomic service 212 can ingest. Similarly, the application sensor data 222 from the application 206 can be fed into the neuroergonomic service 212 via an application programming interface (API) service 216 to be processed by the neuroergonomic service 212 (or processed by a different backend service into a format that can be ingested by the neuroergonomic service 212).

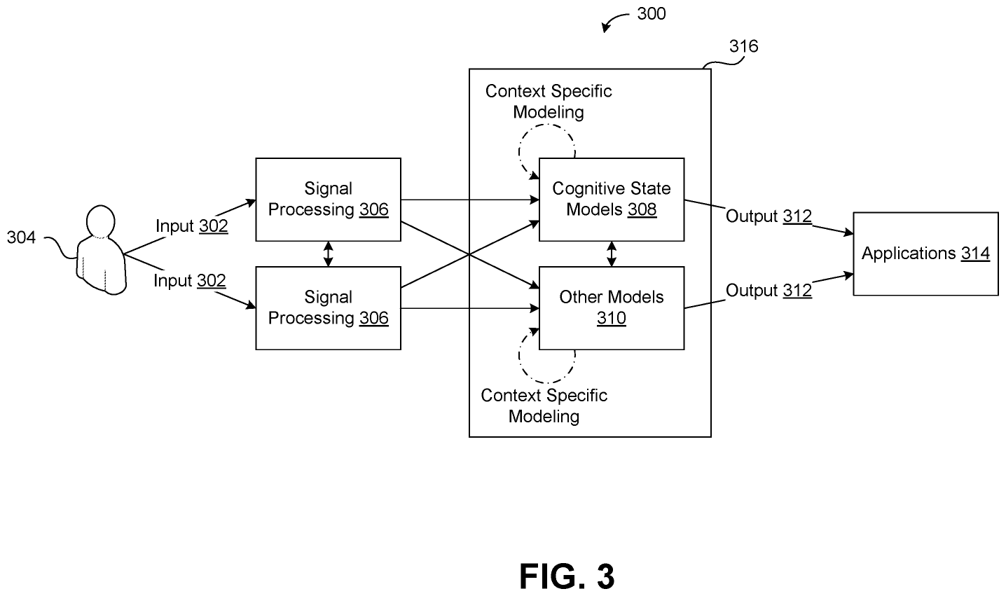

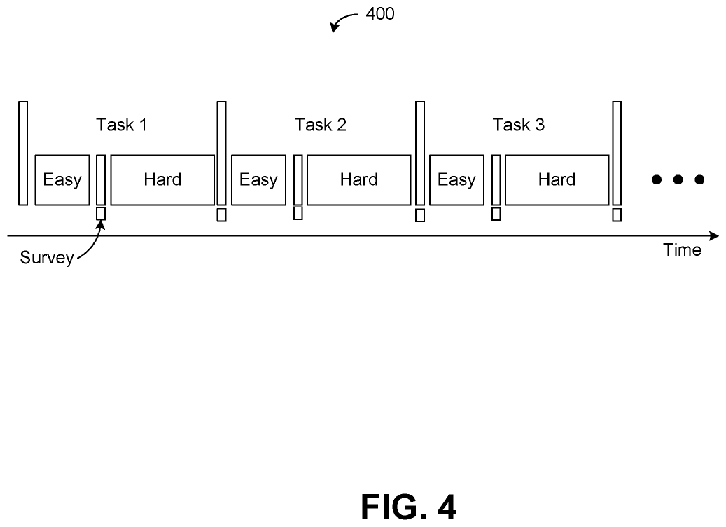

The neuroergonomic service 212 can store the received data and then process them, or simply process them in real time without storing them. The neuroergonomic service 212 includes one or more machine learning models that can take the processed sensor data 214 and/or the application sensor data 222 as multimodal inputs and predict one or more cognitive states 220 of the user 202, if the user 202 permits such use of the sensor data 208. The machine learning models can be trained to infer, for example, cognitive load, stress, affect, attention, engagement, focus, and/or other neuroergonomic measures that may be insightful. Each of these measures may use or be based on one, some, or all of the multimodal sensor data 208 and/or application sensor data 222. The machine learning models will be described in more detail below in connection with FIGS. 3 and 4.

The values of the predicted neuroergonomic measures can be expressed in many different ways. The values can be expressed in a categorical scale (e.g., high stress, medium stress, low stress, or no stress). The values can be expressed in a numerical scale, such as in the real number domain, in the integer domain, using non-negative numbers, or using a normalized scale (e.g., from 0 to 1 or from −1 to 1). The values can include units or be unitless. The values may be descriptive (e.g., relaxed, excited, bored, etc.). The choice of output format can be based on the needs and desires of downstream applications and how users and/or developers prefer to receive the outputs.

The cognitive states 220 of the user 202 that are output by the neuroergonomic service 212 can be input into the application 206 or other downstream applications. The application 206 can access the cognitive states 220 that the neuroergonomic service 212 calculated by making calls to the API service 216. The API service 216 can also provide the application 206 access to the sensor data 208 (e.g., heart rate, EEG signals, pupil size, etc.) and/or application sensor data 222 from other applications.

In some implementations, the application 206 can “pull” the cognitive states 220 and/or the sensor data 208 from the neuroergonomic service 212. For example, the application 206 can query or stream the neuroergonomic measures that indicate the cognitive states 220 of the user 202 through the API service 216. Alternatively, in other implementations, the neuroergonomic service 212 can “push” the cognitive states 220 and/or the sensor data 208 to the application 206. For example, the application 206 can request a notification (e.g., a callback) under certain conditions. For instance, the application 206 can request a notification only if the user's cognitive load is high or above a numerical threshold value. This implementation reduces the burden on computing resources (e.g., CPU, memory, and network usage) compared to having the application 206 frequently poll the neuroergonomic service 212 or having the application 206 constantly stream cognitive state data from the neuroergonomic service 212.

The outputs from the API service 216 can include a user identification (ID). The outputs can also include timestamps associated with the output data. In one implementation, the outputs from the API service 216 may use JavaScript Object Notation (JSON) format or any other format acceptable to the downstream applications.

The application 206 can show the user 202 the input data (i.e., sensor data 208 and/or the application sensor data 222). For example, the application 206 can display a graphical user interface that shows the user's heart rate, breathing rate, pupil size, etc. In one implementation, the user 202 can be shown input data of other users, with their permission, such as the heart rates of other participants in a videoconference or other employees in the user's team or department.

The application 206 can show the user 202 the cognitive states 220 of the user 202. For example, the application 206 can display a graphical user interface that shows the user's cognitive load, her affective state, etc. The application 206 can also display to the user 202 cognitive states of the other users, with their permission, such as the stress level of other employees in a virtual meeting.

Any output format and technique can be used to convey the input data (e.g., the sensor data 208) and/or the output data (e.g., the cognitive states 220) to the user 202, including, for example, graphical output, auditory output, tactile output, etc. For example, a time-series line graph (which would be running in real time) can display the heart rate, a numerical display can show the body temperature, a color-coded indicator can show the stress levels (e.g., red for high stress, yellow for medium stress, and green for low stress), and avatar faces or emoticons can indicate the affective states. The output can show only the current measurement or show historical measures for a certain time window (e.g., the last minute, the last hour, the entire day, etc.). Many other output implementations are possible.

The application 206 can use the cognitive states 220 of the user 202 to enhance user experience. The application 206 can modify the experience of the user 202 based on the sensor data 208 collected by the sensors 204 and/or the application sensors data 222 collected by the application 206.

In some implementations, the neuroergonomic insights can include recommendations for changing the state or condition of the user 202. The neuroergonomic service 212 can include a recommendation module that can determine and output a set of recommendations to the application 206. For example, if the machine learning models in the neuroergonomic service 212 predict that the user 202 is fatigued, then the recommendation module can recommend to the application 206 to dim the screen brightness. Alternatively, if the neuroergonomic service 212 detects that the user 202 is struggling to see the screen (e.g., squinting her eyes and bringing her eyes closer to the screen), then the recommendation module can recommend to the application 206 to increase the brightness of the screen. Therefore, the recommendation module can output recommendations and/or feedback for changing the digital environment that affects the user 202. If the user 202 is in a low attention state, then the recommendation module can recommend that the application 206 (or an operating system) enter a focus mode that reduces or eliminates distractions. The recommendation module can change the user's workflow, change the user's schedule, play ambient sounds or music, increase font size, etc. Many examples of recommendations are possible.

In one implementations, the application 206 can register a particular function with the neuroergonomic service 212, via an API call, such that the function will be executed upon a certain condition being met. The application 206 provides parameters for executing the function, such as a threshold condition that would trigger the execution of the function. For example, the application 206 can register a function that increases the font size to be executed if the user's cognitive load rises to the medium level, and that recommends taking a break if the user's cognitive load rises to the high level.

Furthermore, the recommendation module can also output recommendations and/or feedback to the user 202, which could be presented to the user 202 by the application 206. For example, if the machine learning models in the neuroergonomic service 212 predict that the user 202 (and perhaps others in the user's team) is experiencing high cognitive load, then the recommendation model could recommend to the user 202 that she take a short break from the current task. If the user 202 is experiencing a high level of stress, the recommendation module can suggest activities to reduce stress, such as playing calming music, exercising, eliminating noise, etc. The recommendation module can be implemented using rule-based algorithms and/or machine learning techniques.

The developers of the application 206 (or developers of any downstream applications) can use the outputs from the neuroergonomic service 212 and configure the application 206 to present the outputs to the user 202, present the recommendations to the user 202, and/or take actions in response to the outputs and/or the recommendations. The developers can choose how to present useful insights to the user 202 as well as choose which recommendations to present and/or implement. That is, the recommendation can be automatically executed (and a notice of the change displayed to the user 202), or the recommendation can be presented to the user 202 for acceptance or rejection.

As mentioned above, the outputs from the neuroergonomic service 212 can be used by application developers for neuromarketing. The neuroergonomic service 212 provides insights regarding the cognitive states 220 of the user 202, which can serve as indicators of the user's preferences. With the user's permission, application developers can leverage these marketing signals for advertising purposes. For example, a product shown to the user 202 may elicit physiological responses, including changes in the user's brain signals. Application developers can use the cognitive states 220 from the neuroergonomic service 212 to determine which products the user 202 likes or dislikes, and which advertisements (e.g., the choice of color, pattern, sounds, smell, etc.) pique the interests of the user 202 based on positive or negative physiological responses.

In some implementations, alternatively or additionally, the recommendation module may be part of the application 206. That is, the developers of the application 206 can configure the application 206 to process the sensor data 208 and the cognitive states 220 from the neuroergonomic service 212, and determine a set of recommendations that can be implemented by the application 206 and/or the user 202.

The example neuroergonomic system 200 has been described in connection with FIG. 2 as a cloud-based system. However, other alternative implementations are possible. For example, the neuroergonomic service 212 can be incorporated into the application 206. The application 206 (itself or through the sensors 204) can measure biological, neurological, and physiological conditions of the user 202. The application 206 can use machine learning models to predict the cognitive states 220 of the user 202. Then, the application 206 can make adjustments to the digital environment and/or output the sensor data 208, and/or present recommendations to the user 202. This local implementation may preserve the privacy of the user 202 by not transmitting sensitive user data to a remote service.

### Data Flow

FIG. 3 illustrates an example data flow 300, consistent with some implementations of the present concepts. If a user 304 opts in, one or more modalities of inputs 302 from the user 304 (and/or affecting the user 304) can be sensed by reading different types of measurements using various instrumentalities. Although FIG. 3 shows only two inputs 302 for simplicity, many modalities of inputs 302 can be measured.

For example, the inputs 302 can include various physiological inputs. The user's pupil dilation, facial expression, gaze (i.e., the direction and focus of the user's eyes), skin flushing, heart rate, and breathing rate can be sense by a camera (e.g., a standalone camera, a surveillance camera, a webcam, or a camera built into a laptop or smartglasses). The user's heart rate can be sensed by an ECG sensor (e.g., incorporated into a wearable such as a smartwatch). The user's brain activity can be sensed by an EEG sensor (e.g., incorporated onto a headphone, a VR headset, a helmet, an earphone, or an earbud). The user's vocal tone, speech volume, and choice of words can be sensed by a microphone in conjunction with speech recognition modules. The user's body temperature can be sensed by a thermometer (e.g., built into a smartwatch). The user's breathing rate can be measured by a PPG sensor (e.g., built into a smartwatch) or a webcam. Many types of measurements relating to the user's physical activity and fitness can be sensed by an IMU.

The inputs 302 can include various digital inputs. An application and/or an operating system can detect the digital focus of the user 304 (i.e., the type of activity that the user 304 is engaged in). The application and/or the operating system (OS) can also provide additional telemetry including level of usage (e.g., typing rate, reading rate, browsing speed, scrolling/swiping rate, speaking rate, multitasking rate, clicking/tapping rate, etc.).

The inputs 302 can include various environmental inputs. The lighting surrounding the user 304 (including intensity of light, the number of light sources, the temperature of the light, and the color of light (e.g., warm light color or cool light color)) can be sensed by a camera, an application, and/or Internet of Things (IoT) devices. The sounds surrounding the user 304 (including the type of sound, speech, or music; the volume of sound; and the number of sound sources) can be sensed by a microphone, an application, and/or IoT devices. A global positioning system (GPS) can sense the user's geographical location including elevation. The user's GPS location, in combination with a mapping database, can determine the user's environment, such as whether the user 304 is at her own home, at her usual workplace or at a remote office, in a hotel, in an airport, inside or outside, at a beach, in the mountains, etc. The user's GPS location, in combination with a weather service, can determine the outside temperature, wind speed, precipitation, etc.

The user 304 can opt in or opt out of any set of the different modalities of the inputs 302. However, a more comprehensive set of the inputs 302 will allow more accurate and more comprehensive outputs, including better recommendations.

Some or all of the inputs 302 may undergo signal processing 306. Similar to the data ingest 210 in FIG. 2, the signal processing 306 processes the inputs 302. For example, the signal processing 306 can change the format and/or structure of the inputs 302, filter the inputs 302, perform error checking, and/or perform time alignment. The signal processing 306 can include pipelines that process the inputs 302 to generate features, such as chunks of formatted data that machine learning models 316 can consume. In some scenarios, multiple inputs 302 can be processed jointly. Although FIG. 3 shows only two boxes for the signal processing 306 that process the two examples of inputs 302, the example data flow 300 can include many more signal processing 306 for handling many modalities of the inputs 302. Not all types of the inputs 302 require signal processing 306.

Next, the inputs 302, whether directly or after signal processing 306, are inputted into one or more machine learning models 316. For example, the machine learning models 316 of FIG. 3 can be included in or used by the neuroergonomic service 212 of FIG. 2. The machine learning models 316 in the example data flow 300 include one or more cognitive state models 308 and can also include other models 310. The machine learning models 316 can make predictions about the mental states and/or the physical states of the user 304.

The cognitive state models 308 can take the inputs 302 and assess the cognitive states of the user 304 by predicting one or more neuroergonomic measures to generate outputs 312. Many different implementations of the cognitive state models 308 are possible. For example, one cognitive state model 308 can take in a single-signal input 302 (e.g., from one sensor) and output one neuroergonomic measure (e.g., stress level). One cognitive state model 308 can take in one signal and output multiple neuroergonomic measures. One cognitive state model 308 can take in multiple signal inputs 302 (e.g., from multiple sensors) and output one neuroergonomic measure. One cognitive state model 308 can take in multiple signal inputs 302 (e.g., from multiple sensors) and output multiple neuroergonomic measures. Alternatively, the four possible implementations can be performed by multiple cognitive state models 308. For example, the cognitive state models 308 can include one model for predicting the stress level, another model for predicting the cognitive load, another model for predicting the affective state, another model for predicting the attention level, another model for predicting the level of engagement, another model for predicting fatigue, and so on. Each cognitive state model 308 can use one or more of the inputs 302 to estimate a cognitive state of the user 304. Furthermore, in additional to the inputs 302, one cognitive state model 308 may use the outputs from one or more of the other cognitive state models 308 as inputs.

Although the data flow 300 from the inputs 302 to the outputs 312 are shown in linear fashion in FIG. 3 for simplicity, the data could also flow in loops where the predictions made by the cognitive state models 308 could go back to the pipeline of data that is processed and inputted to the cognitive state models 308. For example, if the cognitive state models 308 initially process the input 302 that includes the user's heart rate signal and detect that the user's heart rate is raised above normal, then the raised hear rate state can be used as feedback to filter and process other input signals 302 (e.g., certain inputs, such as respiration rate and body temperature, become more important than other inputs when the heart rate is high). As another example, the heart rate inputs from multiple users may reveal a statistical bias in the heart rates of a group of people in a specific situation, and the statistical bias can be used as feedback to reprocess the inputs. Thus, the data can go around in loops through the pipeline and the cognitive state models 308 multiple times.

The other models 310 can include one or more custom models. These custom models can be used for a variety of predictions, including higher order predictions. For example, the other models 310 can calculate the life expectancy of the user 304, predict the next word(s) that the user 304 will type or speak, determine the preferences of the user 304, measure the wellbeing of the user 304, be used for empathic sensing (e.g., predicting the user's emotion based on her facial expression), etc.

In some implementations, global models (e.g., cognitive state models or other models) for the general population are trained. The global models can be trained offline using data from a controlled setting and then put into use in the real world, and/or the global models can be trained online in real time and on the fly using real-life data. The global models can be used in all contexts and scenarios, or they may be used as base models (i.e., default and/or initial models) from which context-specific models can be trained. The context-specific models can be updated and fine-tuned to a particular user, a particular use case scenario (e.g., a particular setting), a particular task, a particular input modality, a particular session, and/or any other particular factors. Both global models and context-specific models can continue to be trained using online data to improve their prediction performance. Furthermore, one or more cognitive state models 308 and/or the other models 310 can be trained jointly. For example, predictions made by one machine learning model can be inputs to another machine learning model. Example training of the machine learning models 316 will be discussed below in connection with FIG. 4.

The outputs 312 from the machine learning models 316 can be accessed by applications 314, for example, via APIs. The outputs 312 can include cognitive states predicted by the cognitive state models 308 and/or any other outputs produced by the other models 310. The applications 314 can be programmed to perform a variety of actions based on the outputs 312, including displaying the outputs 312 to the user 304, attempting to change the cognitive states of the user 304, etc.

### Machine Learning Models

The machine learning models that take the inputs (e.g., from sensors measuring the user's body) and make predictions about the user's cognitive states can be trained in two stages: offline and online. The offline stage involves a controlled environment (e.g., a laboratory or any controlled setting) where sensing information can be gathered from participants and/or the environment surrounding the participants. In one implementation, one or more participants can be subjected to test use cases (e.g., perform certain tasks) that mimic real-life experiences in order to purposefully induce certain mental and/or physical states. For example, participants can perform mental mathematical calculations to elevate the cognitive load. The offline inputs from these training tasks can be used to train the machine learning models. Accordingly, when the machine learning models are online, they can make accurate predictions based on real-life online inputs.

FIG. 4 illustrates an example timeline 400 of tasks, consistent with some implementations of the present concepts. In one implementation, the timeline 400 is used in a user study to gather training data for offline training of a stress state model. The timeline 400 can involve multiple participants, multiple tasks, and multiple sessions. For example, in one example user study, one or more participants of specific or varied demographics (e.g., gender, age, race, intelligence, education, body weight, etc.) are given tasks to perform in defined time periods. The selection of the participants can be purposely varied when training global machine learning models for wider applications. The selection of the participants can be intentionally specific when training context-specific machine learning models that target specific users.

One or more modalities of inputs, discussed above, are measured using one or more sensors while the participants are performing certain tasks in a number of sessions. The tasks can vary in difficulty level (e.g., easy, medium, or hard). After each task, the participants are surveyed to gauge their stress level. For example, the stress level can be measured on a binary scale (i.e., stressed or not stressed), a categorical scale (e.g., no stress, low stress, medium stress, or high stress), or a numeric scale (e.g., zero to ten). The participants' evaluation of their own stress levels can be used as labels for the measured sensor data in supervised learning. Alternative and/or additional surveys can be taken, for example, to obtain labels for the participants' cognitive load, attention level, affective state, etc. The participants' answers to the surveys can serve as ground truth labels for sensor inputs collected as training data. Alternatively, rather than taking manual surveys, the participants can be assigned to perform representative tasks that are known to induce certain cognitive states. For example, a task of listening to meditative music is known to reduce stress, whereas a task of complex computational math is known to increase cognitive load. Furthermore, labels can be automatically generated based on the participants' performance in certain tasks. For instance, labels of various levels of cognitive load can be automatically generated based on the number of correct mental math problems answered. Other techniques for automatically generating labels are possible. Thus, the labels can be obtained without surveying the participants. Collecting more training data (e.g., more participants, more modalities of inputs, more volume of data, more variety of tasks, etc.) can improve the offline training of the machine learning models.

In one example session of the user study, as illustrated in FIG. 4, Task 1 involves mental mathematical calculations. A participant starts with 2 minutes of easy math problems, such as “(0×1)+1=?” and then 5 minutes of hard math problems, such as “(19×19)+66=?” In another implementation, the easy math task involves the participants simply counting 1, 2, 3, . . . , and so on. A medium math task involves the participant counting multiples of six (i.e., 6, 12, 18, 24, . . . , and so on). A hard math task involves giving the participant a four-digital number (e.g., 4,253) and instructing the participant to repeatedly subtract a two-digit number (e.g., 34) from the four-digital number. The more difficult math task can induce stress. The difficulty level can be adjusted by using smaller or larger numbers.

Task 2 involves Stroop tests, which are neuropsychological tests that assess the participant's ability to inhibit cognitive interference that occurs when processing one stimulus impedes the simultaneous processing of another stimulus. A common Stroop test involves displaying text using a colored font and asking the participant to identify the color of the font. In an easy Stroop test, the text describes the color of the font (e.g., “red,” “green,” “blue,” “purple,” etc.). It is easier for the participant to identify the font color when the text description matches the font color. In a hard Stroop test, the text describes a color that is different from the font color. For example, the text “red” is displayed using green font color. It is harder for the participant to identify the font color when the text describes a different color.

Task 3 involves instructing the participant to type a caption. An easy Task 3 can be as simple as displaying text of sentences and asking the participant to type the same sentences. A hard Task 3 can be more mentally challenging, such as displaying an image and asking the participant to type three sentences describing the image.

Another example task is the Berg card sorting task (also called Wisconsin card sorting). The participant is given a card and is also presented with a row of additional cards. The cards can have different symbols, different colors, different numbers of items, etc. The participant is instructed to sort the given card by placing it in the row of cards. A variation of this task instead asks the participant to match the given card with one of the additional cards presented. However, in either variation, the participant is not told any rules to use when sorting or matching the cards. If the participant answers correctly, the participant is given a positive feedback. If the participant answers incorrectly, the participant is given a negative feedback. To make the task more difficult, the unknown rule can be changed without any notice to the participant (other than the positive or negative feedback in response to the participant's answers). The unknown rule can be changed with increasing frequency for even higher difficulty level.

The above tasks are examples. Many other varieties of tasks are possible. Tasks that produce detectable changes in the biological, neurological, and/or physiological measurements are more useful and helpful in training accurate machine learning models. The length of the tasks can also vary. Generally, lengthier tasks can induce additional stressors. Also, collecting training data for longer time windows (i.e., sufficiently long sessions of tasks) can provide more meaningful data that the machine learning models can interpret. The tasks can include more variations in difficulty levels. That is, in addition to the easy and hard difficult levels in FIG. 4, three or more difficulty levels can be used. In one implementation, breaks can be added between some or all of the tasks.

While the participants are performing the tasks, multiple modalities of inputs are sensed, measured, and collected. Examples of inputs include RGB videos of the participants, near and far infrared videos of the participants, EEG time series, ECG time series, and/or any of the above-mentioned readings. The multimodal inputs can also be measured during the breaks to form a baseline.

The offline inputs collected during offline training of the machine learning models need not exactly match the online inputs that will be sensed during online use of the machine learning models. Indeed, it is preferrable to collect as much training data as possible to build accurate machine learning models. However, during online use, the machine learning models are capable of making predictions even with a limited set of online inputs. For example, participants in offline training may be more willing (or were required) to wear a cap-type EEG headgear and a chest trap heart rate monitor, whereas an ordinary online user in everyday environment may be unwilling to purchase and wear such cumbersome and expensive devices. Thus, a 32-channel EEG sensor can be worn by participants during offline training of the machine learning models, but even if an online user wears only a single-channel (alpha) in-ear EEG sensor or no EEG sensor at all, the machine learning models can use the limited input data to make predictions about the cognitive states of the user. This capability of the machine learning models to take only one or few online inputs and still predict a range of cognitive states will be explained further below.

The offline input data collected can be used as training data to train one or more machine learning models. Accordingly, the machine learning models may discover and learn through analyzing the training data that one or more of the input measurements correlate with (or are indicators of) one or more of the cognitive states. In one example, the training data may reveal that a set of features (e.g., EEG bands, pupil size, heart rate, etc.) can be used by a machine learning model to accurately predict the stress level and the cognitive load provided by the participants through the surveys by modeling classification problems: {stressed, not stressed} and {no cognitive load, low cognitive load, medium cognitive load, high cognitive load]. As another example, the collected EEG data may reveal that the participants generated stronger delta band signals during the hard versions of the math, Stroop, and caption tasks; weaker alpha band signals during the hard versions of the math, Stroop, and caption tasks; stronger theta band signals during the hard versions of the math and Stroop tasks; and prominent upper beta band signals during the hard versions of the math and caption tasks. As yet another example, the machine learning models may learn the relationship between the pupil size and cognitive load, or may determine that there is no relationship at all. Many other modalities of inputs can be collected during offline training to determine whether they are distinguishing features that can be used to train the machine learning models to predict one or more cognitive states. Furthermore, different training data sets may reveal different relationships among the sensed measurement inputs and the cognitive state outputs. A technical advantage of the present concepts is that researchers (e.g., physiologists and/or neurologists) need not manually discover the exact relationships among the multiple modalities of sensed inputs and the several cognitive states through research, testing, trial-and-error, etc. Instead, consistent with the present concepts, machine learning models can automatically assess the training data to learn the functions that can predict the cognitive states based on the multimodal inputs.

Collecting multiple modalities of input signals has technical advantages, because the signals can help improve each other's predictive power. Human bodies do not operate in isolation. For example, if a person's heart rate rises, then some other aspects of the person's body (including the mind) can change correspondingly, and vice versa. Therefore, rather than using a single input signal to make predictions, using multiple input signals is a more holistic approach to essentially modeling the user's bodily functions and interactions, and then predicting the cognitive states of the user. The predictive capability of the machine learning models can be a function of the number of input signal available not only for training but also during online use.

In some implementations, the cognitive state models are prediction models, similar to machine learning models that predict the weather. Weather prediction models take in environmental inputs, such as temperature, humidity, wind speed, wind direction, date and time, etc., and forecast future weather conditions, such as temperature, precipitation, etc. Here, the cognitive state models take in multimodal input signals, such as heart rate, EEG bands, respiration rate, pupil size, body temperature, etc., and predict the cognitive states, such as cognitive load, stress, affect, attention level, etc.

Many different varieties of machine learning model types can be employed in the present concepts to build the cognitive state models. Experimental tests of building machine learning models, consistent with the present concepts, were performed by the inventors. The inventors experimented with many different types of machine learning models. The experimental tests involved using at least the below described list of machine learning models and artificial intelligence techniques to build the cognitive state models.

For example, a support-vector machine (SVM) is a supervised learning model with learning algorithms that analyze data for classification and regression analysis. The objective of the SVM algorithm is to find a hyperplane in an N-dimensional space (where N is the number of features) that distinctly classifies (i.e., separates) the data points. Although relatively simple, SVMs were shown to be successful during the experiments of the present concepts.

A deep learning neural network uses multiple layers in the network, including an input layer, multiple hidden layers, and an output layer. The hidden layers extract features that dictate the results (i.e., picks out the features that improve performance). Learning can be supervised, semi-supervised, self-supervised, or unsupervised. In some implementations of the present concepts, a classifier model includes an output layer that outputs a classification of a cognitive state (e.g., no stress, low stress, medium stress, or high stress). In other implementations of the present concepts, the neural network includes a regression layer that outputs a numerical value that characterizes the magnitude of a particular cognitive state (e.g., the cognitive load value on a scale of zero to one).

A convolutional neural network (CNN) includes layers that use convolutions. A convolution is a mathematical operation that takes two functions and produces a third function that expressed how the shape of one function is modified by the other function. CNNs are feed-forward neural networks that use filters and pooling layers to learn more and more of the features of the data with each passing layer. CNNs are conventionally designed to process two-dimensional data, such as images.

A one-dimensional convolutional neural network (1D CNN) is useful for processing one-dimensions data, such as time series data. 1D CNN uses one-dimensional arrays for the kernels and the feature maps. In a 1D CNN, the kernel slides along only one dimension. 1D CNNs perform one-dimensional convolutions (e.g., scalar multiplications and additions). 1D CNNs were successful during the experiments of the present concepts.

A non-exhaustive list of additional machine learning models and techniques that were used during the experimental tests to build the cognitive state models include: linear discriminant analysis (LDA), logistic regression, decision tree or decision forest, singular value decomposition (SVD), k-nearest neighbor (KNN), multilayer perceptron (MLP), long short-term memory (LSTM), encoding-based algorithms, autoencoder, variational autoencoder (VAE), transformer encoder, ensemble, correlation-based time series of graph (TSG), online training, fine-tuning, and lifelong learning.

Another example, U.S. patent application Ser. No. 17/553,063, filed on Dec. 16, 2021 (attorney docket no. 410746-US-NP), describes a technique involving accessing a multivariate time series set of samples collected by multiple biological sensors sensing a first biological function over a first period of time, dividing the data set into windows, calculating statistical dependencies between the samples of the timeseries data collected by each sensor, generating a relationship matrix as a function of the statistical dependencies, and transforming the relationship matrix to generate a first feature vector for each window of time that captures the statistical dependencies amongst the sensors that can be used in a prediction model. The '063 application, entitled “A Statistical Dependence-Aware Biological Predictive System,” is incorporated herein by reference in its entirety.

Any of the above choices of specific machine learning model type and/or machine learning technique can be employed, or they may be employed in combination. Furthermore, the choice can depend on the type and amount of available training data, the type of available input signals, specific tasks that the online users will be performing, the type of cognitive states being predicted, etc., particularly because different machine learning models and techniques have advantages and disadvantages.

The relationship among the multiple modalities of inputs and the multiple cognitive states is complex. A technical advantage of the present concepts is that the complex relationship can be learned using artificial intelligence. There is no need to manually determine the relationship by trying to fit and adjust express functions. In some implementations, the machine learning models may use known relationships. For example, the neurophysiological community has discovered that EEG signals with alpha frontal asymmetry is a good indicator of stress. This discovery can be used as a feature that is fed into the machine learning models used in the present concepts.

Another technical advantage of the present concepts is that the machine learning models, when put into online use, do not need all of the multimodal inputs to predict useful outputs. Some or even many of the inputs may be missing, for example, because the user did not subscribe or did not voluntarily opt into having certain measurements taken. Nevertheless, the machine learning models of the present concepts can generate at least some of the desired outputs. In some scenarios, the machine learning models use one or more types of inputs to infer one or more other types of inputs, because the different modalities of inputs are not necessarily independent of each other. Different aspects of the human body work together, are regulated together, and are inter-related, as explained above.

Depending on the types of models and the cognitive states being predicted, one or more types of inputs may be necessary to predict a specific cognitive state. Thus, depending on which input modalities are available while online, a specific set of outputs will be available from the machine learning models. Generally, more outputs will be available if more inputs are available. On the flip side, one or more modalities of inputs may be unnecessary to predict a specific cognitive state, either because the machine learning model learned that those input modalities do not affect the specific cognitive state or because the machine learning model learned to infer those input modalities from other available inputs. Accordingly, even though offline training of the machine learning models generally involves collecting many modalities of offline inputs (e.g., as many modalities as possible), the machine learning models can still operate with a smaller set of online input modalities and generate useful neuroergonomic insights.

Furthermore, the human body functions and responds similarly while performing different tasks. For example, the certain sensor readings from a user's body may be similar whether the user is stressed from writing for stressed from math problems. Moreover, human bodies function and respond similarly even among different people. For example, certain sensor readings from one person can be similar to those certain sensors readings from another person if both people are experiencing high cognitive load. Thus, the machine learning models, consistent with the present concepts, are transferable among different tasks and/or among different users. That is, the training data, which were obtained from the specific participants who performed specific tasks during offline training and used to train the machine learning models, can be leveraged across different tasks and across different users to accurately predict the cognitive states of many people performing various tasks during online use of the machine learning models. For example, the sensor readings obtained during training while offline participants performed math problems, Stroop tests, and caption writing can be used to predict cognitive states while online users are reading the news, playing games, chatting in forums, videoconferencing, etc. Accordingly, the machine learning models are useful not only for (1) same task, same person, but are also useful for (2) same task, different people, (3) different tasks, same person, and (4) different tasks, different people.

Having more participants perform more types of tasks during offline training will help train the machine learning models to better predict cognitive states during online use when a wide variety of users are performing a myriad of different tasks. However, training data is often limited to a small set of participants performing a small set of tasks (e.g., due to limited resources, volunteers, and time). Therefore, the offline participants cannot cover all types of online users, the offline tasks cannot cover all types of online tasks (i.e., use cases) that the users will perform, and the offline settings cannot cover all types of online settings in which the machine learning models will be used. That is, there are far too many demographics of users, too many tasks (e.g., typing, studying, problem solving, browsing, etc.), and too many settings (e.g., office, home, airport, train, hotel, etc.) for offline training to fully capture.

Accordingly, the performance of the machine learning models can be improved through online training that adapts the models to the specific use case scenarios, i.e., to specific users, specific tasks, and/or specific settings. Online training involves gathering online input data (e.g., real time input data) as the user uses the machine learning models and then training the machine learning models using the online input data. The online training data can be used to further train and improve the general machine learning models. Additionally or alternatively, the online training data can be used to train individualized machine learning models. Individualized machine learning models can be trained by taking the offline trained machine learning models and then using the online training data to fine-tune the offline trained machine learning models. Alternatively or additionally, individualized machine learning models can be combined together with the offline trained machine learning models in an ensemble to produce improved predictions.

Online training can be supervised and unsupervised. Supervised online training tends to provide better results. Labels for the online training data can be obtained by surveys (e.g., stressed or not stressed) if online users are willing to provide such survey-like feedback. Otherwise, labels for the online training data can be obtained by asking online users to perform certain representative tasks that are known to induce specific cognitive states.

Unsupervised or self-supervised learning can be performed. For example, an online sensor signal is captured, and the online sensor signal is processed to get it as close to the offline training data as possible by the machine learning models. The processing can involve extractions, transformations, compressions, removing artifacts, etc. Furthermore, one or more signals (e.g., digital signals or environmental signals) may provide some feedback to the machine learning models. For example, if the user is feeling stressed and turns off the screen to take a break, that action can be a feedback signal to the machine learning models that the user is experiencing high stress level. Other user actions, user conditions, and/or environmental conditions (such as typing very fast, idling, dimming the room lighting, etc.) can serve as feedback that indicate the user's cognitive state.

For both offline training and online training, more training data will improve the performance of the machine learning models. If vast amounts of training data are collected (for example, from millions of users worldwide for five years), then accurate general machine learning models can be built and used by the general population for a wide range of tasks. However, if such general machine learning models are not available due to the limited amount of training data, then individualized machine learning models can be trained through fine-tuning processes for specific users, specific tasks, and/or specific settings.

### Processes

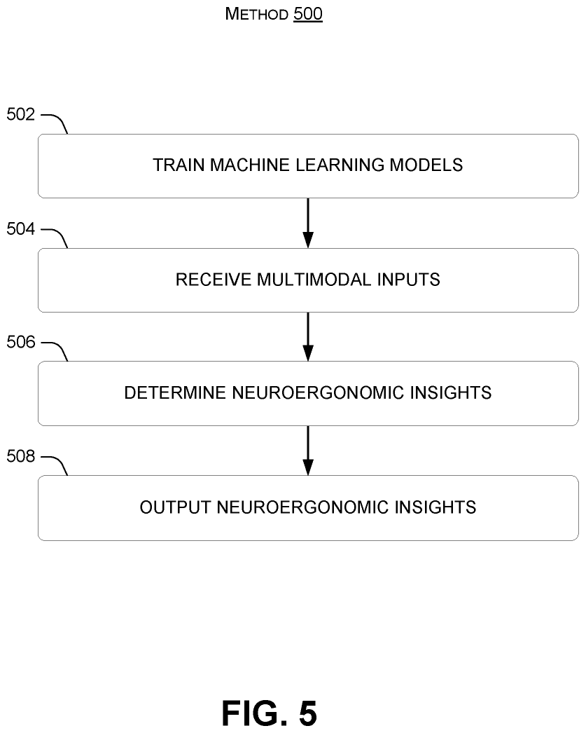

FIG. 5 illustrates an example neuroergonomic method 500, consistent with some implementations of the present concepts. The neuroergonomic method 500 is presented for illustration purposes and is not meant to be exhaustive or limiting. The acts in the neuroergonomic method 500 may be performed in the order presented, in a different order, or in parallel or simultaneously, may be omitted, and may include intermediary acts therebetween.

In act 502, machine learning models are trained. The machine learning models can be trained to provide neuroergonomic insights. For example, the machine learning models include cognitive state models that can predict cognitive states of a user. The machine learning models can be trained offline using training data obtained through participants performing a set of tasks and sensing relevant measurements. The machine learning models can also continue to be trained after the machine learning models are deployed and go online.

In act 504, multimodal inputs are received. The multimodal inputs can include physiological inputs from the user, digital inputs from an application and/or the user, and/or environmental inputs affecting the user. The multimodal inputs can be obtained through sensors that contact the user, non-contact sensors, software applications, hardware devices, and/or databases.

In act 506, neuroergonomic insights are determined based on the multimodal inputs. In one implementation, machine learning models can take the multimodal inputs and predict one or more neuroergonomic insights. For example, neuroergonomic insights can include the measured inputs, cognitive states of the user, and/or recommendations for improving the user's health (including mental health), satisfaction, and/or productivity.

In act 508, the neuroergonomic insights are outputted. In one implementation, the neuroergonomic insights are outputted to an application, for example, via APIs. The application can display the neuroergonomic insights to the user. Alternatively or additionally, the application can take certain actions based on the neuroergonomic insights. For example, the application can change the screen brightness, reschedule a meeting, increase the font size, play soothing ambient music, change the digital focus, etc.

Act 502 may be a continual process of further training, adapting, fine-tuning, and/or refining the machine learning models based on new signals. The new signals may be based on the user's actions, such as taking breaks when cognitive load is high, playing music when stress is high, etc. The new signals can continue to train the general machine learning models for everyone as well as fine-tune the individualized machine learning models for the specific user.

### Configurations

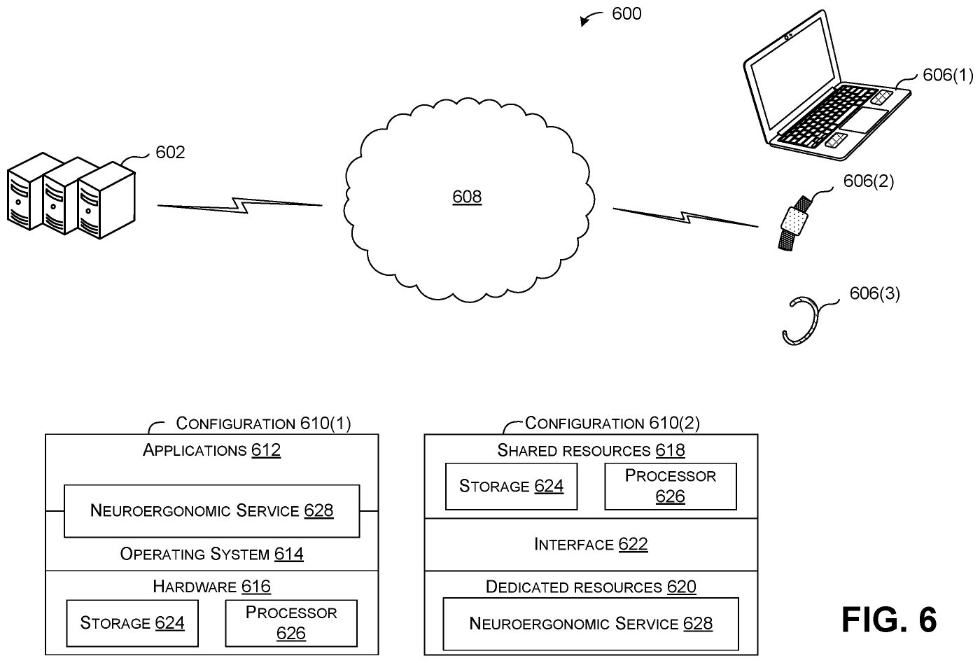

FIG. 6 illustrates an example configuration of a neuroergonomic system 600, consistent with some implementations of the present concepts. This example neuroergonomic system 600 includes sensors 606 for taking measurement inputs associated with a user. For example, a laptop 606(1) includes a camera, a microphone, a keyboard, a touchpad, a touchscreen, an operating system, and applications for capturing physiological inputs, digital inputs, and/or environmental inputs associated with the user. A smartwatch 606(2) includes biosensors for capturing the heart rate, respiration rate, perspiration rate, etc. An EEG sensor 606(3) measures brain activity of the user. The sensors 606 shown in FIG. 6 are mere examples. Many other types of sensors can be used to take readings that relate to or affect the neuroergonomic measurements that are desired.

The measured inputs are transferred to a neuroergonomic server 602 through a network 608. The network 608 can include multiple networks and/or may include the Internet. The network 608 can be wired and/or wireless.

In one implementation, the neuroergonomic server 602 includes one or more server computers. The neuroergonomic server 602 runs a neuroergonomic service that takes the inputs from the sensors 606 and outputs neuroergonomic insights. For example, the neuroergonomic service uses machine learning models to predict the cognitive states of the user based on the multimodal inputs from the sensors 606. For example, the neuroergonomic service may perform the neuroergonomic method 500. The outputs from the neuroergonomic service can be accessed via one or more APIs. The outputs can be accessed in other ways besides APIs. Furthermore, a software development kit (SDK) may be available for software developers to build and configure applications to use the outputs from the neuroergonomic service and display the outputs, perform other actions based on the outputs to change the cognitive states of the user, and/or provide additional inputs to the neuroergonomic service (e.g., to further train the machine learning models).

Although FIG. 6 shows the neuroergonomic service as a cloud-based service and being hosted on the neuroergonomic server 602, other configurations are possible. For example, the neuroergonomic service can run on a user computer, a laptop, or a smartphone, or even incorporated into an end-user application.

FIG. 6 also shows two example device configurations 610 of the neuroergonomic server 602. The first device configuration 610(1) represents an operating system (OS) centric configuration. The second device configuration 610(2) represents a system on chip (SoC) configuration. The first device configuration 610(1) can be organized into one or more applications 612, an operating system 614, and hardware 616. The second device configuration 610(2) can be organized into shared resources 618, dedicated resources 620, and an interface 622 therebetween.

The device configurations 610 can include a storage 624 and a processor 626. The device configurations 610 can also include a neuroergonomic service 628. For example, the neuroergonomic service 628 uses machine learning models to predict cognitive states of the user.

As mentioned above, the second device configuration 610(2) can be thought of as an SoC-type design. In such a case, functionality provided by the device can be integrated on a single SoC or multiple coupled SoCs. One or more processors 626 can be configured to coordinate with shared resources 618, such as storage 624, etc., and/or one or more dedicated resources 620, such as hardware blocks configured to perform certain specific functionality.

The term “device,” “computer,” or “computing device” as used herein can mean any type of device that has some amount of processing capability and/or storage capability. Processing capability can be provided by one or more hardware processors that can execute data in the form of computer-readable instructions to provide a functionality. The term “processor” as used herein can refer to central processing units (CPUs), graphical processing units (GPUs), controllers, microcontrollers, processor cores, or other types of processing devices. Data, such as computer-readable instructions and/or user-related data, can be stored on storage, such as storage that can be internal or external to the device. The storage can include any one or more of volatile or non-volatile memory, hard drives, flash storage devices, optical storage devices (e.g., CDs, DVDs etc.), and/or remote storage (e.g., cloud-based storage), among others. As used herein, the term “computer-readable media” can include transitory propagating signals. In contrast, the term “computer-readable storage media” excludes transitory propagating signals.

Generally, any of the functions described herein can be implemented using software, firmware, hardware (e.g., fixed-logic circuitry), or a combination of these implementations. The term “component” as used herein generally represents software, firmware, hardware, whole devices or networks, or a combination thereof. In the case of a software implementation, for instance, these may represent program code that performs specified tasks when executed on a processor (e.g., CPU or CPUs). The program code can be stored in one or more computer-readable memory devices, such as computer-readable storage media. The features and techniques of the component are platform-independent, meaning that they can be implemented on a variety of commercial computing platforms having a variety of processing configurations.

### Technical Advantages

In addition to the technical advantages discussed above, the present concepts are able to provide predictions for neuroergonomic measures using robust machine learning models that integrate multiple sensing modalities. The wide breadth of sensing modalities can include physiological (EEG, ECG, etc.), digital (gaze, facial responses, application telemetry, etc.), and/or environmental (lighting, sounds, etc.). By considering multiple modalities of inputs, the machine learning models can more accurately predict the cognitive states of users.

The machine learning models translate the multimodal sensing to cognitive states for applications at scale in an extensible way. The present concepts also abstract the domain expertise typically needed to interact with different input signals. Because the neuroergonomic service provides modular, expert-informed, pre-trained models as building blocks, application developers can rapidly build custom solutions without needing extensive subject-matter expertise or relevant data.

The neuroergonomic service can be deployed as a cloud service to enable security and scale. User-centric privacy controls enable protection of personal data use and provide trust that generated measures are not used without the user's permission and/or knowledge.

The neuroergonomic service are useful in providing neuroergonomic insights even with only a limited number of inputs, because the neuroergonomic service can leverage its training gained from a wide array of inputs collected. That is, the user can provide as much or little sensing modalities as she is comfortable. For example, a user may be willing to provide only a limited set of inputs (e.g., only her heart rate, pupil dilation, or perspiration rate). The user may be unwilling or unable to share a myriad of inputs, either for privacy reasons or because she does not wish to incur the cost of purchasing multiple sensors. The neuroergonomic services, however, can use the limited inputs to provide useful neuroergonomic insights, because the machine learning models may have learned relationships between the inputs that are available and inputs that are unavailable. In this sense, the neuroergonomic service can be, to some degree, sensing modality ambivalent. Thus, the neuroergonomic service is not dependent on a particular modality of input or a particular sensor. And, the neuroergonomic service provides cross-compatibility between the wide array of available sensor devices and applications that can react to neuroergonomic measures.

The present concepts can provide application developers and/or users with actionable recommendations for improving user's cognitive states, mental health, physical health, productivity, satisfaction, and/or mood. The neuroergonomic system can also automatically take actions based on the neuroergonomic insights and/or recommendations to seamlessly benefit the user's wellbeing. Thus, the present concepts can benefit not only the individual users but also their managers, employers, administrators, etc.

The present concepts provide a set of models and algorithms that fuse multiple input data sources to provide high quality neuroergonomic predictors. The APIs enable downstream applications to integrate with the neuroergonomic predictors. The present concepts also provide a platform upon which software developers can build specialized models and exchange those models with other developers.

### Additional Examples

Various examples are described above. Additional examples are described below. One example includes a system comprising a processor and a storage including instructions which, when executed by the processor, cause the processor to: receive online multimodal inputs from at least one sensor that senses multiple conditions of a user, predict one or more cognitive states of the user based on the online multimodal inputs by using a machine learning model, and output the one or more cognitive states to an application.

Another example can include any of the above and/or below examples where the at least one sensor includes a camera, a microphone, an electrocardiogram (ECG) sensor, an electroencephalogram (EEG) sensor, a keyboard, a mouse, a touchscreen, an application, or an operating system.

Another example can include any of the above and/or below examples where the one or more cognitive states include a cognitive load level, a stress level, an affective state, or an attention level.

Another example can include any of the above and/or below examples where the instructions further cause the processor to determine a recommendation based on the one or more cognitive states of the user and output the recommendation to the application.

Another example can include any of the above and/or below examples where the instructions further cause the processor to receive training data including offline multimodal inputs that are labeled with cognitive states and offline train the machine learning model using the training data.

Another example can include any of the above and/or below examples where the offline multimodal inputs are associated with participants performing tasks that include a plurality of difficulty levels.

Another example can include any of the above and/or below examples where the instructions further cause the processor to online train the machine learning model using the online multimodal inputs.

Another example includes a method comprising receiving online multimodal inputs associated with a user, determining a neuroergonomic insight for the user using a machine learning model, and outputting the neuroergonomic insight to an application.

Another example can include any of the above and/or below examples where the online multimodal inputs include physiological inputs, digital inputs, or environmental inputs.

Another example can include any of the above and/or below examples where the online multimodal inputs include a heart rate, a pupil size, an electroencephalogram (EEG), a respiration rate, a perspiration rate, or a body temperature.

Another example can include any of the above and/or below examples where the neuroergonomic insight includes one or more cognitive states of the user.

Another example can include any of the above and/or below examples where the one or more cognitive states include a cognitive load, a stress level, an affective state, or an attention level.

Another example can include any of the above and/or below examples where the method further comprises training the machine learning model using training data including offline multimodal inputs.

Another example can include any of the above and/or below examples where the method further comprises collecting the training data by having participants perform tasks and conducting surveys of the participants' cognitive states.

Another example can include any of the above and/or below examples where a number of online multimodal inputs is less than a number of offline multimodal inputs.

Another example can include any of the above and/or below examples where the neuroergonomic insight includes a recommendation for changing a cognitive state of the user.

Another example can include any of the above and/or below examples where the recommendation includes scheduling a meeting, changing a screen brightness, changing a font size, taking a break, changing an ambient sound, or changing an ambient lighting.

Another example includes a computer readable storage medium including instructions which, when executed by a processor, cause the processor to: provide application data to a neuroergonomic service, the neuroergonomic service using machine learning models trained to predict cognitive states of a user based on the application data and sensor data, receive the cognitive states of the user from the neuroergonomic service, and take an action for changing a particular cognitive state of the user.

Another example can include any of the above and/or below examples where the instructions further cause the processor to determine whether the particular cognitive state satisfies a threshold condition, where the action is taken in response to determining that the particular cognitive state satisfies the threshold condition.

Another example can include any of the above and/or below examples where the action includes one or more of: recommending a break, changing a font size, or changing a display brightness.

1. A system, comprising:
a processor; and
a storage including instructions which, when executed by the processor, cause the processor to:
receive online multimodal inputs from at least one sensor that senses multiple conditions of a user;
predict one or more cognitive states of the user based on the online multimodal inputs by using a machine learning model; and
output the one or more cognitive states to an application.

2. The system of claim 1, wherein the at least one sensor includes a camera, a microphone, an electrocardiogram (ECG) sensor, an electroencephalogram (EEG) sensor, a keyboard, a mouse, a touchscreen, an application, or an operating system.

3. The system of claim 1, wherein the one or more cognitive states include a cognitive load level, a stress level, an affective state, or an attention level.

4. The system of claim 1, wherein the instructions further cause the processor to:
determine a recommendation based on the one or more cognitive states of the user; and
output the recommendation to the application.

5. The system of claim 1, wherein the instructions further cause the processor to:
receive training data including offline multimodal inputs that are labeled with cognitive states; and
offline train the machine learning model using the training data.

6. The system of claim 5, wherein the offline multimodal inputs are associated with participants performing tasks that include a plurality of difficulty levels.

7. The system of claim 1, wherein the instructions further cause the processor to:
online train the machine learning model using the online multimodal inputs.

8. A method, comprising
receiving online multimodal inputs associated with a user;
determining a neuroergonomic insight for the user using a machine learning model; and
outputting the neuroergonomic insight to an application.

9. The method of claim 8, wherein the online multimodal inputs include physiological inputs, digital inputs, or environmental inputs.

10. The method of claim 8, wherein the online multimodal inputs include a heart rate, a pupil size, an electroencephalogram (EEG), a respiration rate, a perspiration rate, or a body temperature.

11. The method of claim 8, wherein the neuroergonomic insight includes one or more cognitive states of the user.

12. The method of claim 11, wherein the one or more cognitive states include a cognitive load, a stress level, an affective state, or an attention level.

13. The method of claim 8, further comprising:
training the machine learning model using training data including offline multimodal inputs.

14. The method of claim 13, further comprising:
collecting the training data by having participants perform tasks and conducting surveys of the participants' cognitive states.

15. The method of claim 13, wherein a number of online multimodal inputs is less than a number of offline multimodal inputs.

16. The method of claim 8, wherein the neuroergonomic insight includes a recommendation for changing a cognitive state of the user.

17. The method of claim 16, wherein the recommendation includes scheduling a meeting, changing a screen brightness, changing a font size, taking a break, changing an ambient sound, or changing an ambient lighting.

18. A computer readable storage medium including instructions which, when executed by a processor, cause the processor to:
provide application data to a neuroergonomic service, the neuroergonomic service using machine learning models trained to predict cognitive states of a user based on the application data and sensor data;
receive the cognitive states of the user from the neuroergonomic service; and
take an action for changing a particular cognitive state of the user.

19. The computer readable storage medium of claim 18, wherein the instructions further cause the processor to:
determine whether the particular cognitive state satisfies a threshold condition,
wherein the action is taken in response to determining that the particular cognitive state satisfies the threshold condition.

20. The computer readable storage medium of claim 18, wherein the action includes one or more of: recommending a break, changing a font size, or changing a display brightness.
